(19) United States
(12) Patent Application Publication
(10) Pub. No.: US 2015/0182757 A1
(43) Pub. Date: Jul. 2, 2015

(54) A METHOD AND SYSTEM FOR COSMETIC SKIN PROCEDURES FOR HOME USE

(71) Applicant: Syneron Beauty LTD, Yoqneam IIIit (IL)

(72) Inventors: Baruch Levine, Afula (IL); Omry Hayet, Ramat Gan (IL); Lion Flyash, Nazaret Illit (IL)

(21) Appl. No.: 14/423,434

(22) PCT Filed: Sep. 30, 2013
(86) PCT No.: PCT/IL13/00074

§ 371 Date: Feb. 24, 2015

Related U.S. Application Data
(60) Provisional application No. 61/712,883, filed on Oct. 12, 2012.

(51) Int. Cl. A61N 5/06; A61B 19/00; A61H 23/00; A61B 18/18

(52) U.S. Cl. CPC A61N 5/0616; A61B 18/18; A61B 19/5212; A61H 23/00; A61N 2005/0626; A61B 2018/00452

(57) ABSTRACT

System for personal skin procedure including one or more portable computers with a camera, a number of software applications (Apps) run on the computer, one or more electromagnetic energy emitting home-use cosmetic treatment devices and one or more communication links between the portable computer and the cosmetic treatment device, the App are configured to employ the computer to analyze and process an image of a segment of skin and extract from the image information regarding the segment of skin as well as determine specific optimal procedure doses of optical energy for treated skin fractions within the segment of skin based on the extracted information.

### TECHNICAL FIELD

The current method and system relate to systems and methods for cosmetic procedures and in particular to methods and systems for cosmetic procedures for skin.

### BACKGROUND

External appearance is important to practically every person. In recent years, methods and systems have been developed for different cosmetic procedures. These cosmetic procedures include wrinkle removal, skin rejuvenation, skin resurfacing, hair removal, treatment pigmented lesions, treatment of vascular lesions and others. In some of these cosmetic procedures, the skin and tissue are treated by one or more types of electromagnetic radiation such as optical illumination (light) and radio frequency (RF).

In recent years, there has been a tendency to move application of cosmetic procedures out of professional clinics and into the home environment where users can carry out the cosmetic procedure in the privacy of their own home.

This tendency also challenges manufacturers of home-use devices for cosmetic procedures to make their products sufficiently safe so that they can be operated by non-professional users, convenient and easy to use and accessible as well as affordable for the average user.

Over recent years portable computers such as laptop computers, tablet computers and mobile phones (smartphones) have become more than communication and text editing devices and more and more of the device functions are being used as tools or components of other, external tools primarily through applications, nicknamed “Apps” that provide an interface code between the portable computer and the desired function or associated tool.

### SUMMARY

The present system and method seek to provide cosmetic system and method that enable control of operation of home-use devices for cosmetic procedures applied to the skin of a non-professional user employing readily available portable computers such as laptop computers, tablet computers and mobile phones (smartphones) using applications (Apps) that provide an interface code between the portable computer and the desired function or associated tool.

There is thus provided in accordance with an example, a system including a portable computer having a display, a camera and a processor operative to process an image of a segment of skin captured by the camera and communicate with a transceiver and a wireless or wired communication link between transceiver in the computer and either a remote computer or a controller inside a home-use cosmetic skin treatment device.

In accordance with another example, the computer could also include a cosmetic skin procedures software application (App), which could be acquired directly from a personal computer or downloaded from the internet and be operative to convert the computer into a data acquiring and processing component of home-use cosmetic skin treatment device.

In accordance with yet another example, the system the portable computer could be a mobile phone or tablet computer including a processor running a cosmetic skin procedures software application (App) to capture an image of a segment of skin, analyze and process the image and extract from the image information regarding the segment of skin, determine specific optimal procedure doses of electromagnetic or mechanical energy for one or more skin fractions within the segment of skin based on the extracted information and change settings of a electromagnetic or mechanical energy emitting home-use cosmetic skin treatment device based on the determined specific optimal procedure doses of electromagnetic energy.

In accordance with still another example, the system App could activate a light energy emitting home-use cosmetic treatment device based on the determined specific optimal procedure doses of light (optical) energy.

In accordance with another example, the system could also include an opaque, semi-opaque or transparent patch adhereable to skin of a user, the patch having one or more of a scale bar, a scale frame, an image identifier and skin pigmentation references.

In accordance with yet another example, there is also provided a method of operation of a home-use devices for cosmetic procedures applied to the skin of a non-professional user employing readily available portable computers such as laptop computers including using a mobile phone or a tablet computer camera and capturing an image of a segment of skin desired to be treated, analyzing and processing the image and extracting from the image information regarding one or more parts of the segment of skin and determining specific optimal procedure doses of electromagnetic or mechanical energy for one or more skin fractions within the segment of skin based on the extracted information.

In accordance with still another example, the method also includes displaying an image of the one or more parts of a segment of skin on a display and storing the image in memory of the mobile phone or tablet computer.

In accordance with another example, the method also includes monitoring progress of a cosmetic procedure by capturing a first image of a segment of skin to be treated, applying a desired cosmetic skin treatment procedure to the segment of skin, capturing a second image of the same segment of skin following application of the desired cosmetic skin treatment procedure to the segment of skin and comparing the second captured image of the segment of skin to the first captured image of the same segment of skin to derive information regarding the progress of the cosmetic skin treatment procedure.

In accordance with still another example the method also includes manually entering into the electromagnetic energy emitting home-use cosmetic skin treatment device the specific optimal doses of electromagnetic energy for a desired skin treatment.

### BRIEF DESCRIPTION OF THE DRAWINGS

The present method and system will be understood and appreciated more fully from the following detailed description, taken in conjunction with the drawings in which:

FIG. 1 is a block diagram of an system and method in accordance with an example;

FIG. 2 is a side view simplified semi-block illustration of a method of application of a home-use cosmetic treatment device in accordance with an example;

FIG. 3A is a simplified illustration of a portable computer application (App) display in accordance with an example;

FIG. 3B a simplified illustration of a captured image as viewed in real time on a portable computer display in accordance with an example;

FIG. 4A is a simplified illustration of a portable computer display in accordance with another example;

FIG. 4B is a simplified illustration of a captured image as viewed in real time on the computer display of FIG. 4A in accordance with another example;

FIG. 4C is a simplified illustration of a portable computer display in accordance with yet another example;

FIG. 4D is a simplified illustration of a captured image as viewed in real time on the computer display of FIG. 4C in accordance with still another example;

FIG. 5A is a plan-view simplified illustration of a patch to be used in a method of application of a home-use cosmetic treatment device in accordance with an example;

FIG. 5B is a side-view and perspective-view simplified illustration of a method of application of a patch in use of a home-use cosmetic treatment device in accordance with an example;

FIG. 6 is a side-view and perspective-view simplified illustration of a method of application of a patch in use of a home-use cosmetic treatment device in accordance with another example;

FIG. 7 is a side-view and perspective-view simplified illustration of a method of application of a patch in use of a home-use cosmetic treatment device in accordance with yet another example;

FIG. 8 is a block diagram illustrating method of use of a home-use cosmetic treatment device in accordance with an example; and

FIG. 9 is a block diagram illustrating method of use of home-use cosmetic treatment device in accordance with another example.

### DETAILED DESCRIPTION

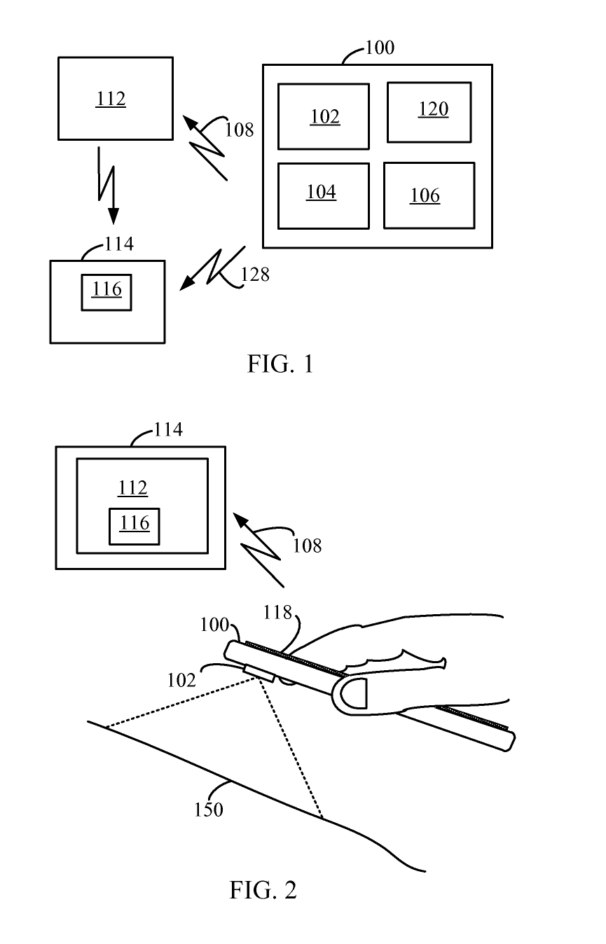

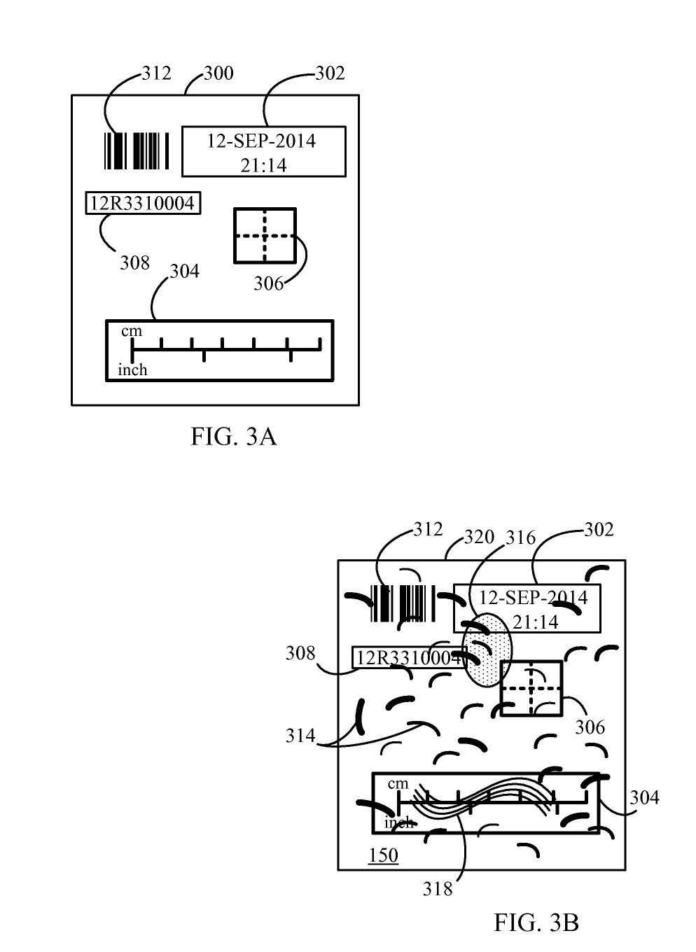

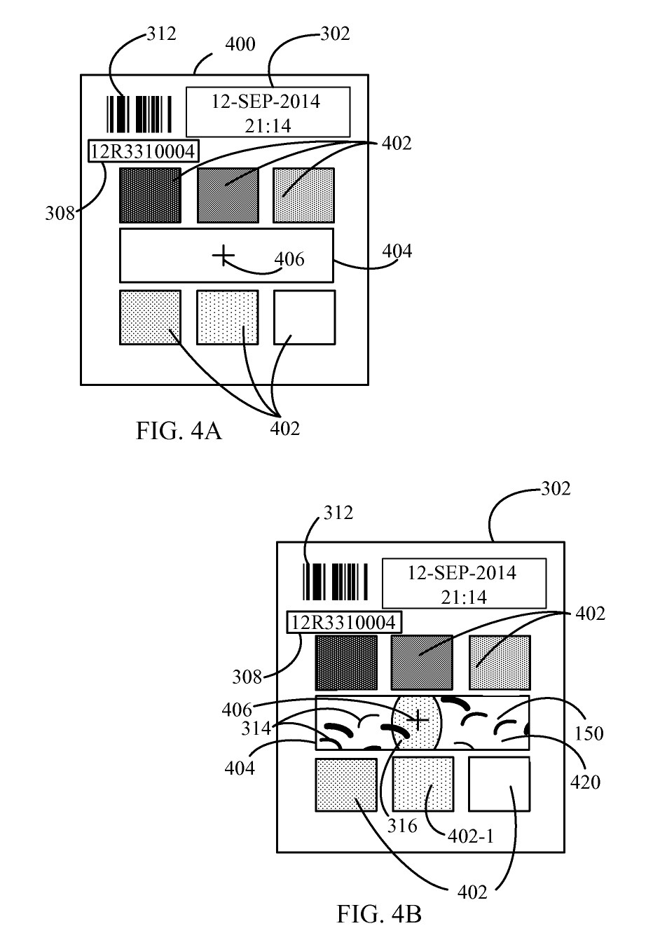

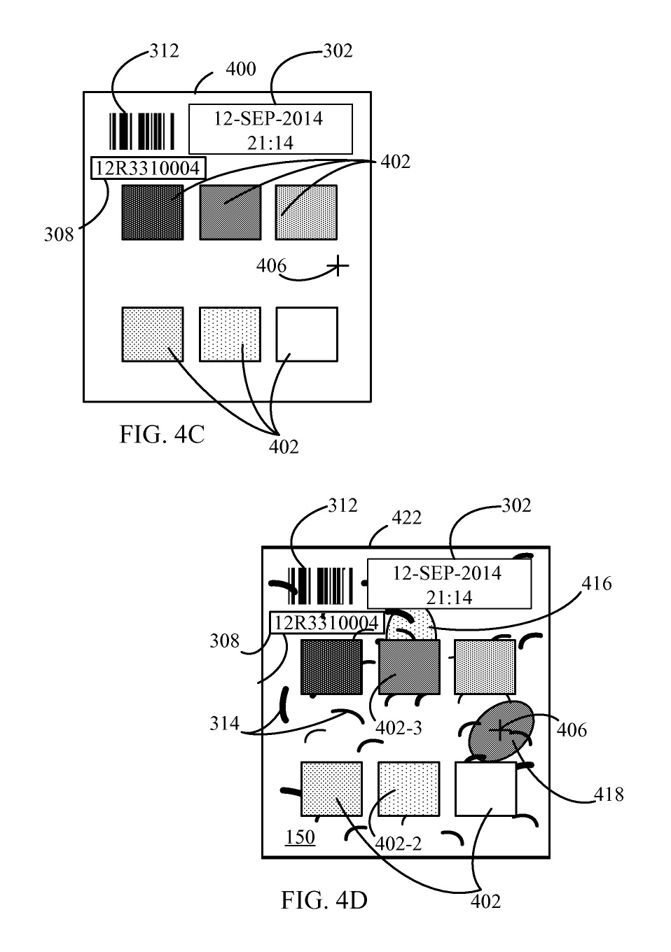

Referring now to FIG. 1, which is a block diagram of a system and method in accordance with an example. A portable computer 100 could include a camera 102, a processor 104 configured to process an image 320 (FIG. 3B), 420 (4B) or 422 (FIG. 4D) captured by camera 102 and communicate with a transceiver 106 and a wireless or wired communication link 108 between transceiver 106 in computer 100 and either a remote computer 112 or a controller 116 inside a home-use cosmetic skin treatment device 114.

Portable computer 100 could also include a touch activated display screen 118.

Computer 100 could also include a cosmetic skin procedures software application (App) 120, which could be acquired directly from a personal computer (not shown) or downloaded from the Internet and be configured to convert computer 100 into a data acquiring and processing component of home-use cosmetic skin treatment device 114 as will be described in greater detail below.

As shown in FIG. 2, which is a side view simplified semi-block illustration of a method of application of device 100 in accordance with an example, portable computer 100, running App 120 (FIG. 1), can be used for capturing an image, such as image 320, 420 and/or 422 of a segment of skin 150 as expressed by dotted lines. Portable computer 102 could be any mobile device including a camera 106, a processor 104 having a memory and a communication link 108 with a remote device similar to device 100 or remote computer 112. Examples for computer 100 could be, but not limited to, a laptop computer, a tablet computer and a smartphone.

Remote computer 112 could be external to and in communication with a home-use cosmetic skin treatment device 114 controller 116 (FIG. 1) or be included in home-use cosmetic skin treatment device 114 alongside or incorporating controller 116 (FIG. 2). Home-use cosmetic skin treatment device 114 could be a cosmetic device for application of electromagnetic or mechanical energy to conduct cosmetic procedures such as, but not limited to, hair removal, pigmented lesions removal and similar. Controller 116 could be, for example a switch controlling dose, duration and similar parameters of the electromagnetic or mechanical energy application to skin segment 150. Such electromagnetic and/or mechanical energy could include electromagnetic and/or mechanical energy commonly used for cosmetic skin treatments such as, but not be limited to optical energy, electrical energy, heat energy, vibration energy and acoustical energy.

Captured image 320, 420 and/or 422 captured by camera 102 could be communicated to processor 104, which in turn could analyze and extract information from image 320, 420 and/or 422 and determine specific optimal treatment doses of electromagnetic energy to be applied to one or more skin fractions (not shown) within segment of skin 150 based on the extracted information. One or more of captured image 320, 420 and/or 422, the extracted information and determined specific optimal treatment doses of electromagnetic energy could be stored in processor 104 memory.

Additionally or alternatively, processor 104 could communicate to remote computer 112 a raw captured image, such as image 320, 420 and/or 422, analyzed and extract information from image 320, 420 and/or 422 and/or determined specific optimal treatment doses of electromagnetic energy to be applied to one or more skin fractions within the segment of skin based on the extracted information, thus changing the settings of cosmetic device 114.

Additionally or alternatively, processor 104 could analyze and extract information from image 320, 420 and/or 422 and determine specific optimal treatment doses of electromagnetic energy to be applied to one or more skin fractions within the segment of skin based on the extracted information and communicate determined specific optimal treatment doses of electromagnetic energy directly to home-use cosmetic skin treatment device 114 controller 116 thus activating cosmetic device 114 based on the determined specific optimal treatment doses of electromagnetic energy.

Reference is now made to FIG. 3A, which is a simplified illustration of a portable computer 100 App 120 display 300 in accordance with an example. Display 300 could include display components such as a time code 302, a scale bar 304 and/or scale frame 306 and an image identifier such as a serial number 308 or barcode 310. All display components could be included any time the image is displayed or printed.

Scale-bar 304 could be used for measuring, for example, linear dimensions of blemishes such as, for example, wrinkles, or length of a hair, whereas scale frame 306 could be used for measuring round or similar blemishes dimensions such as pigmented lesions or specific limited areas covered with hair. Measurement and measurement input could be done manually by a user. Alternatively, measurements and dimensions of various parts of skin segment 150 such as scars, wrinkles, hairs or pigmented lesions appearing in an image such as image 320, 420 and/or 422 (FIG. 4) could be derived automatically from the image and processed by device 100 processor 104.

Additionally and optionally, portable computer 100 App 120 display 300 could also include a time code indicating a time of day and/or date at which an image is captured so that to enable logging images captured by, for example, camera 106 or documenting a sequence in which a series of images were captured for future reference.

FIG. 3B depicts a simplified illustration of a captured image 320 as viewed in real time on display 300 using App 120 (FIG. 1) in accordance with an example. A user, viewing the captured image of skin segment 150 via display 300 can manipulate device 100 so that to align image 320 scale bar 304 and/or scale frame 306 with one or more parts of skin segment 150 desired to be treated such as hairs 314, one or more pigmented lesions 316, scars and/or one or more wrinkles 318.

Captured image 320, including displayed identifiers, scales and timecode could be stored in processor 104 memory or, alternatively processed by processor 104 as described above and transmitted to remote computer 112 or directly to cosmetic device 114 controller 116.

Alternatively, a user viewing the captured image of skin segment 150 via display 300 can manipulate device 100 so that to capture one or more parts of skin segment 150 desired to be treated such as hairs 314, one or more pigmented lesions 316, scars and/or one or more wrinkles 318. Processor 104 can derive from the captured image sizes and dimensions of one or more parts of skin segment 150 desired to be treated as well as home-use cosmetic skin treatment device 114 evaluate other information such as parameters selected from a group of parameters including number of hairs, density of hair, pigment of hair, length and thickness of hair and location of hairs within skin segment 150, pigment parameters of the desired area to be treated and parameters of electromagnetic energy to be applied, such as type of electromagnetic energy, dose, duration of application and similar. In this configuration, a device 100 display, such as display 300 (FIG. 3A) would display only a time code and/or other information not appearing on display 300.

Processor 104 (FIG. 1) could analyze and extract information regarding the parameters of one or more parts of skin segment 150 such as the parameters described above captured in image 320 and determine specific optimal skin treatment doses, e.g., hair treatment doses, of electromagnetic energy to be applied to one or more skin fractions (not shown) within segment of skin 150 (FIG. 2) based on the extracted information.

Alternatively and optionally, device 100 could employ App 120 to communicate the determined specific optimal hair treatment doses of electromagnetic energy to a hair-removal home-use cosmetic skin treatment device 114 controller 116 and to activate cosmetic device 114.

Reference is now made to FIG. 4A, which is a simplified illustration of a portable computer 100 App 120 (FIG. 1) display 400 in accordance with another example. As shown in FIG. 4A, App 120 could include a display for cosmetic treatment of a pigmented lesion 316 (FIGS. 3B and 4B). Similar to display 300 (FIG. 3A), display 400 could also include display components such as a timecode 302 and an image identifier such as a serial number 308 or barcode 310. Additionally, display 400 could also include additional display components such as skin pigmentation or skin tone references 402 flanking a window 404 made of a transparent material. A cross-hairs guide 406 could be printed on window 404.

Alternatively and optionally, processor 104 could extract from a memory associated with it one or more display elements including scales such as, for example, scale bar 304 or scale frame 306 and skin pigmentation or skin tone references 402. The displayed elements could be superimposed on the captured image display 300/400, for example, in the peripheral area of the captured image display 300/400 so that not to block the view of the captured image. Different computers/devices could have different types of displays and the color tones of the reproduced captured image may vary between computers/devices and not necessary be a faithful representation of the actual pigmentation and/or tone of the segment of skin as appears in the captured image. ICC color profiling techniques developed by International Color Consortium® (http://www.color.org) could be applied to each display to ensure faithful color reproduction.

FIG. 4B is a simplified illustration of a captured image 420, as viewed in real time on display 400 window 404 using App 120 in accordance with another example. A user can manipulate device 100 so that to align cross-hairs guide 406 with a desired area to be treated such as pigmented lesion 316. The user, employing touch screen 118 (FIG. 2) can select a pigmentation reference 402 closest in color to the color of the desired area to be treated in skin segment 150, by pressing on the selected reference 402. In FIG. 4B, the pigmentation reference that appears to be closest in color to desired area to be treated pigmented lesion 316 is pigmentation reference 402-1.

Processor 104 (FIG. 1) could analyze and extract information regarding the pigment parameters of the desired area to be treated (e.g., pigmented lesion 316) captured in image 420 and determine specific optimal pigment lesion removal treatment doses of electromagnetic energy to be applied to one or more skin fractions (not shown) within pigmented lesion 316 containing segment of skin 150 based on the extracted information.

Alternatively and optionally, device 100 could employ App 120 (FIG. 1) to communicate the determined specific optimal pigment lesion removal treatment doses of electromagnetic energy to a pigment treatment home-use cosmetic skin treatment device 114 controller 116 (FIGS. 1 and 2) and to activate cosmetic device 114.

Alternatively and optionally and as shown in FIG. 4C, which is a simplified illustration of a portable computer 100 App 120 (FIG. 1) display 400 in accordance with yet another example, App 120 could include a display for cosmetic treatment of a pigmented lesion without a window 404 (FIG. 4A). Cross-hairs guide 406 could be located at any location in App 120 (FIG. 1) display 400 and could be manipulated and movable within display 400 as desired by a user. In this configuration, the user can bring cross-hairs over several desired areas to be treated such as pigmented lesions 416 and 418 (FIG. 4D) as will be described in greater detail below.

FIG. 4D is a simplified illustration of a captured image 422 as viewed in real time on display 400 (FIG. 4C) using App 120 (FIG. 1) in accordance with still another example. A user can drag cross-hairs guide 406 over a first desired area to be treated such as, for example, pigmented lesion 416 and mark the location by, for example, double clicking cross-hairs guide 406. The user, employing touch screen 118 (FIG. 2) can select a pigmentation reference 402 closest in color to the color of the desired area to be treated in skin segment 150 (FIG. 2), by pressing on the selected reference 402.

Alternatively, a user viewing the captured image of skin segment 150 via display 400 can manipulate device 100 so that to capture one or more parts of skin segment 150 desired to be treated such as one or more pigmented lesions 416/418. Processor 104 (FIG. 1) can derive from the captured image pigment color tone as well as sizes and dimensions of one or more pigmented lesions 416/418 of skin segment 150 desired to be treated as well as evaluate other information such as parameters selected from a group of parameters including parameters of electromagnetic energy to be applied, such as type of electromagnetic energy, dose, duration of application and similar. In this configuration, a device 100 display, such as display 400 (FIGS. 4A and 4C) would display only a time code and/or other information not appearing on display 400.

The process can be repeated as many times as desired by the user dragging cross-hairs guide 406 over a second desired area to be treated such as, for example, pigmented lesion 418 and marking the location by, for example, double clicking cross-hairs guide 406. The user, employing touch screen 118 (FIG. 2) can select a pigmentation reference 402 closest in color to the color of the desired area to be treated in skin segment 150, by pressing on the selected reference 402.

In FIG. 4D, the pigmentation reference that appears to be closest in color to desired area to be treated pigmented lesion 416 is pigmentation reference 402-2 and the pigmentation reference that appears to be closest in color to desired area to be treated pigmented lesion 418 is pigmentation reference 402-3.

Processor 104 could analyze and extract information regarding the pigment parameters of the desired area to be treated (e.g., pigmented lesions 416 or 418) captured in image 422 and determine specific optimal pigment lesion removal treatment doses of electromagnetic energy to be applied to each of one or more skin fractions (not shown) within pigmented lesions 416 and/or 418 containing segment of skin 150 based on the extracted information.

Alternatively and optionally, device 100 could employ App 120 to communicate the determined specific optimal pigment lesion removal treatment doses of electromagnetic energy of each of the pigmented lesions to be treated to a pigment treatment home-use cosmetic skin treatment device 114 controller 116 (FIGS. 1 and 2) and to activate cosmetic device 114 (FIGS. 1 and 2).

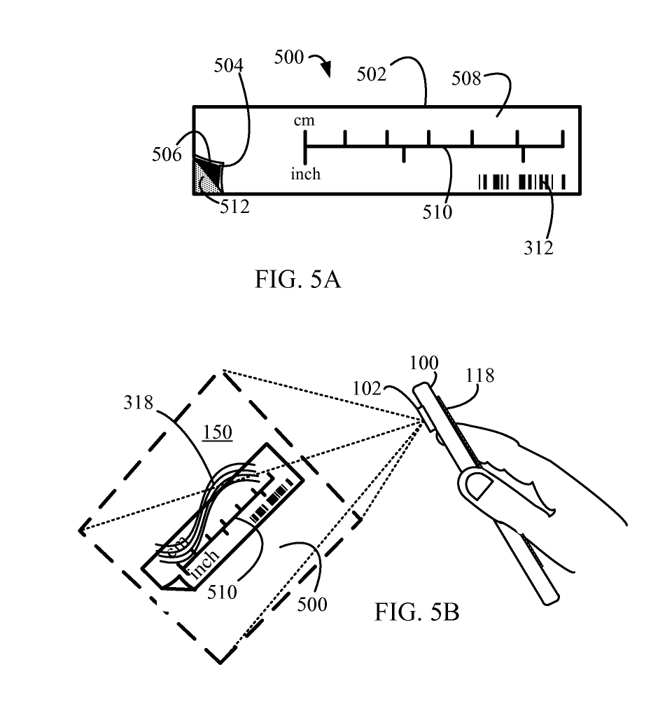

Referring now To FIG. 5A, which is a plan-view simplified illustration of a patch to be used in a method of application of device 100 in accordance with an example. Patch 500 could be a sticker made of a sheet of a biocompatible resilient material 502 such as paper, nylon, plastic, synthetic or natural fabric or similar, a first surface thereof 504 coated with a biocompatible adhesive creating a sticky layer 506 easily adherable to a biological surface such as skin segment 150. A second surface 508 on the opposite side of patch 500, opposing first surface 504, could include a print of a scale-bar 510 such as scale bar 304 (FIG. 3A). A peelable protective backing layer 512 could cover sticky layer 506 and be removed before application of patch 500 to skin segment 150. Patch 500 could also carry an identifier such as, for example, a barcode 312.

Patch 500 could be opaque, semi-opaque or transparent and be used for measuring, for example, linear or other blemishes such as wrinkles, as depicted in FIG. 5B or length of a hair. Use of Patch 500 obviates the need for a scale such as scale 304 (FIG. 3A) to be displayed by App 120, in which case a display, such as display 300 (FIG. 3A) would display only a time code and/or other information not appearing on patch 500.

Referring now to FIG. 5B, which is a side-view and perspective-view simplified illustration of a method of application of patch 500 in use of device 100 in accordance with an example. Patch 500 could be adhered to a segment of skin 150, demarcated in FIG. 5B with broken lines by peeling backing layer 512 to expose sticky layer 506 and applying sticky layer 506 to skin segment 150 so that printed second surface 508 faces away from skin segment 150. In FIG. 5B, patch 500 is transparent and including an imprint of scale-bar 510 such as scale bar 304 (FIG. 3A). The transparent patch could be made of a material selected not to affect the accuracy of information derived from a captured image. Patch 500 could be placed so that scale 510 is adjacent to, or as depicted in FIG. 5B, overlying a lesion desired to be treated, which in FIG. 5B is depicted by a wrinkle 318.

A user can activate camera 102 and capture an image of skin segment 150 such as images 320 (FIG. 3B) or 422 (FIG. 4D) and including patch 500 as expressed by dotted lines. The image could be processed and communicated to remote computer 112 or controller 116 of home-use cosmetic skin treatment device 114 as explained above.

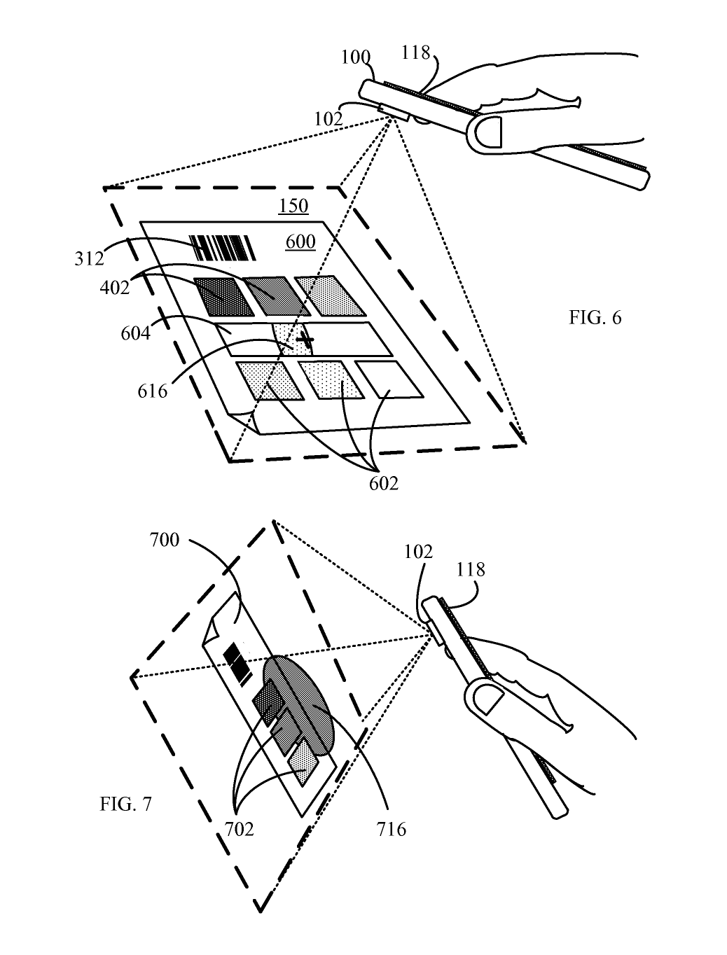

Reference is now made to FIG. 6, which is a side-view and perspective-view simplified illustration of a method of application of patch 600 in use of device 100 in accordance with another example. A patch 600, similar in construction to patch 500 of FIG. 5 could additionally include an aperture 604, which could be made of a resilient biocompatible clear material and carry an imprint of a cross-hairs guide 406. Alternatively and optionally, aperture 604 could be a cutout in patch 600, in which case patch 600 would not include cross-hairs guide 406.

Patch 600 could also include skin pigmentation references 602 similar to references 402 of FIGS. 4A and 4B flanking aperture 604. Patch 600 could also carry an identifier such as, for example, a barcode 312.

Use of Patch 600 obviates the need for skin pigmentation references such as skin pigmentation references 402 (FIGS. 4A and 4C) to be displayed by App 120, in which case a display, such as display 400 (FIG. 3A) would display only a time code and/or other information not appearing on patch 600.

Patch 600 could be adhered to a segment of skin 150, demarcated in FIG. 6 with broken lines, in a fashion similar to that described in FIG. 5. Patch 600 could be placed so that a pigmented lesion 616 desired to be treated appears in aperture 604, flanked by skin pigmentation or skin tone references 402.

A user can activate camera 102 and capture an image as expressed by dotted lines of skin segment 150 such as images 320 (FIG. 3B) or 422 (FIG. 4D) and including patch 600 and pigmented lesion 616. The image could be processed and communicated to remote computer 112 or controller 116 of home-use cosmetic skin treatment device 114 as explained above.

Reference is now made to FIG. 7, which is a side-view and perspective-view simplified illustration of a method of application of patch 700 in use of device 100 in accordance with yet another example.

A patch 700, similar in construction to patch 500 of FIG. 5 could additionally include skin pigmentation references 702 similar to references 402 of FIGS. 4A, 4B and 6, arranged on a patch similar to patch 500 (FIG. 5). Patch 700 could also carry an identifier such as, for example, a barcode 312. In FIG. 7, patch 700 is transparent.

Patch 700 could be adhered to a segment of skin 150, demarcated in FIG. 7 with broken lines, in a fashion similar to that described in FIG. 5. Patch 700 could be placed adjacent or alongside to, or as depicted in FIG. 7 partially overlaying a pigmented lesion 716 desired to be treated so that skin pigmentation or skin tone references 402 can be easily compared to pigmented lesion 716. (The skin pigmentation or skin tone references have been selected according to known Fitzpatrick Classification Scale http://dermatology.about.com/od/cosmeticprocedure/a/fitzpatrick.htm.)

A user can activate camera 102 and capture an image as expressed by dotted lines of skin segment 150 such as images 320 (FIG. 3B) or 422 (FIG. 4D) and including patch 700 and pigmented lesion 716. The image could be processed and communicated to remote computer 112 or controller 116 of home-use cosmetic skin treatment device 114 as explained above.

As explained above, patch 500 could be used as a scale for measurement of dimensions such as length, width, diameter and comparison of relative sizes of hairs and blemishes (e.g., scars, wrinkles and pigmented lesions). Patches 600 and 700 could be used for skin pigmentation or skin tone determination (e.g., to supplement hair-removal optical energy treatment), level of skin or lesion pigmentation, hair pigmentation and similar.

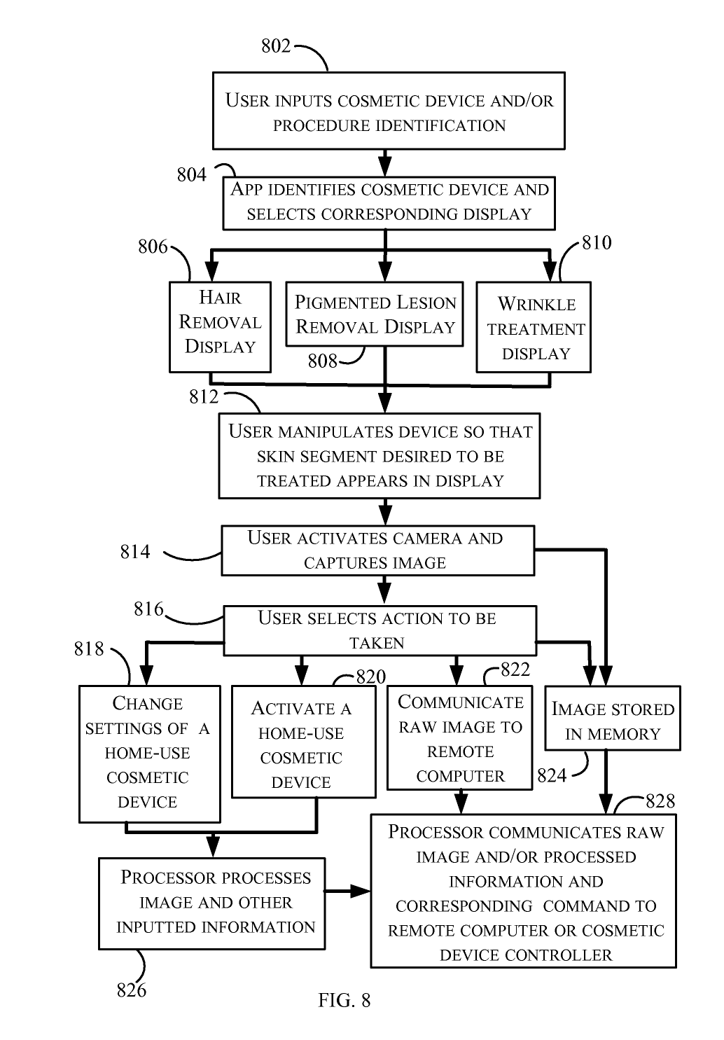

Reference is now made to FIG. 8, which is a block diagram illustrating method of use of device 100 App 120 in accordance with an example. A user could input (block 802) into device 100 a home-use cosmetic skin treatment device 114 identification by, for example, inputting home-use cosmetic skin treatment device 114 serial number, model number or designated identification number or by selecting a type of device 114 from a roll-down table provided by App 120. Alternatively and optionally, the user could scan a barcode (if provided) on home-use cosmetic skin treatment device 114 and have App 120 automatically identify the cosmetic device. Alternatively and optionally, device 100 could be physically connected to home-use cosmetic skin treatment device 114 by a designated communication cable such as a USB connection cable or similar or using a known wireless communication protocol such as for example, Bluetooth. Alternatively and optionally, the user can input a desired procedure to be performed selected from, for example, a roll-down list of cosmetic procedures.

App 120 could identify home-use cosmetic skin treatment device 114 and select a display corresponding to the home-use cosmetic skin treatment device 114 or cosmetic procedure to be performed (block 804) such as, for example, a hair removal display (block 806) similar, for example, to display 300 (FIG. 3A), a pigmented lesion removal display (block 808) similar, for example to display 400 (FIG. 4A) or a combination of displays 300 and 400 or similar or a wrinkle treatment display (block 810) such as, for example display 300 (FIG. 3A). It will be appreciated by those skilled in the art that the App 120 displays described above are disclosed as display examples only and App 120 displays in practice could include additional and/or other display components or combinations thereof.

Once App 120 displays the relevant display on touch activated display screen 118 (FIG. 2), user could manipulate device 100 so that a skin segment 150 desired to be treated appears in display on screen 118 (block 812). User then could activate camera 102 (FIG. 2) and capture an image such as image 320 (FIG. 3B), 420 (4B) or 422 (FIG. 4D) of skin segment 150 (FIG. 2) desired to be treated (block 814).

Once the image is captured App 120 could request the user to select an action to be taken (block 816) selected from a group of displayed options:
Activate home-use cosmetic skin treatment device 114 (block 820);
Change settings of the identified home-use cosmetic skin treatment device 114 and apply a recommended skin treatment protocol derived as a result of captured image 320 processing; (block 818);
and/or
Communicate the raw image to remote computer 112 (FIG. 1) (block 822).

In some examples a mobile phone or tablet computer camera could capture an image of a segment of skin and process the captured image to derive specific optimal doses of electromagnetic energy for a desired skin treatment as described above. The skin treatment settings (i.e., specific optimal doses of electromagnetic energy) being a result of captured image processing could be displayed on the mobile phone or tablet computer screen in a form of a set of instructions for the user. The user could enter manually into the electromagnetic energy emitting home-use cosmetic skin treatment device the specific optimal doses of electromagnetic energy for a desired skin treatment followed by the home-use cosmetic skin treatment device applying the electromagnetic energy emitting home-use cosmetic skin treatment device to perform a desired skin treatment based on the derived specific optimal doses of electromagnetic energy.

Additionally or alternatively, user could select to store the captured image in device 100 processor 104 memory (block 824). Additionally or alternatively, the raw image could be stored automatically following activation of camera 102 and the capturing of the image of skin segment 150 (block 814).

When user selects at least one of to change settings or activate home-use cosmetic skin treatment device 114, processor 104 could process the captured image and other inputted information such as described above, including hair parameters selected from a group of parameters including number of hairs, density of hair, pigment of hair, length and thickness of hair and location of hairs within skin segment 150, pigment parameters of the desired area to be treated and parameters of electromagnetic energy to be applied to skin segment 150 (block 826) and communicate (block 828) the processed information to remote computer 112 and/or controller 116. Additionally or alternatively, processor 104 could communicate the captured raw image (block 814) to remote computer 112 (block 928).

Alternatively and optionally, processor 104 of device 100 could monitor progress of a cosmetic procedure by comparing a currently captured image of the segment of skin (block 814) with an image of the same segment of skin, retrieved from processor 104 memory, captured prior to application of the current procedure or during a previous procedure and stored in memory of the mobile phone or tablet computer processor 104 (block 824). Information extracted from the comparison could be integrated and processed with the parameters described above (block 828).

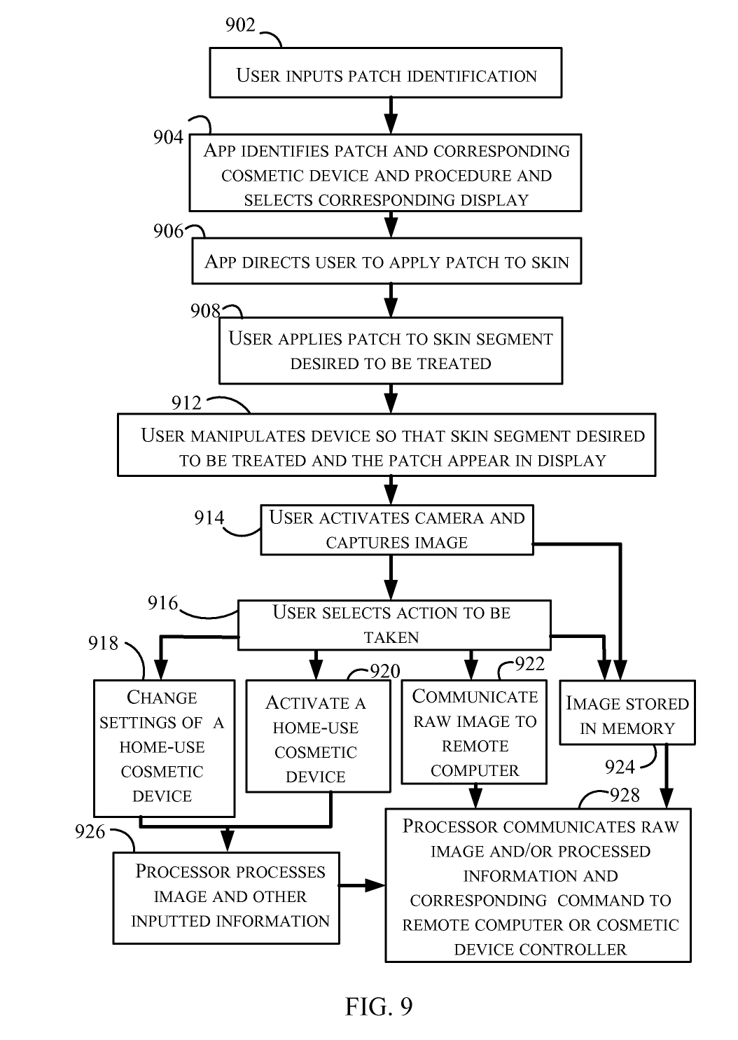

Reference is now made to FIG. 9, which is a block diagram illustrating method of use of device 100 App 120 in accordance with another example. A user could input into device 100 a patch 500/600/700 identification (block 902) by, for example, inputting patch 500/600/700 serial number, designated identification number or by selecting a type of patch 500/600/700 from a roll-down table provided by App 120. Alternatively and optionally, the user could scan a patch 500/600/700 identifier such as a barcode on patch 500/600/700 and have App 120 automatically identify a home-use cosmetic skin treatment device 114 associated with the specific patch 500/600/700. Alternatively and optionally, device 100 could be physically connected to a home-use cosmetic skin treatment device 114 associated with the specific patch 500/600/700 by a designated communication cable such as a USB connection cable or similar. Alternatively and optionally, the user can input a desired procedure to be performed selected from, for example, a roll-down list of cosmetic procedures.

App 120 could identify a home-use cosmetic skin treatment device 114 that corresponds to the identified patch 500/600/700 (block 904) and, optionally, enable activation of a home-use cosmetic skin treatment device 114 only if App 120 identified device 114 to be the correct home-use cosmetic device associated with the inputted patch 500/600/700 identification information.

Once patch 500/600/700 and corresponding home-use cosmetic skin treatment device 114 are identified, App 120 could direct the user to apply the patch to a skin segment 150 desired to be treated (block 906) followed by the user applying the patch to skin segment 150 (908).

User could manipulate device 100 so that a skin segment 150 desired to be treated including patch 500/600/700 appears in display on screen 118 (block 912). User then could activate camera 102 (FIG. 2) and capture an image such as image of skin segment 150 (FIG. 2) desired to be treated together with patch 500/600/700 (block 914).

Once the image is captured App 120 could request the user to select an action to be taken (block 916) selected from a group of displayed options:
Activate home-use cosmetic skin treatment device 114 (block 920); and/or
Apply settings of the identified home-use cosmetic skin treatment device 114 (block 918);
Communicate the raw image to remote computer 112 (FIG. 1) (block 922).

Additionally or alternatively, user could select to store the captured image in device 100 processor 104 memory (block 924). Additionally or alternatively, the raw image could be stored automatically following activation of camera 102 and the capturing of the image of skin segment 150 (block 914).

When user selects at least one of to change settings or activate home-use cosmetic skin treatment device 114, processor 104 could process the captured image and other inputted information such as described above, including hair parameters selected from a group of parameters including number of hairs, density of hair, pigment of hair, length and thickness of hair and location of hairs within skin segment 150, pigment parameters of the desired area to be treated and parameters of electromagnetic energy to be applied to skin segment 150 (block 926) and communicate (block 928) the processed information to remote computer 112 and/or controller 116. Additionally or alternatively, processor 104 could communicate the captured raw image (block 914) to remote computer 112 (block 928).

Alternatively and optionally, processor 104 of device 100 could monitor progress of a cosmetic procedure by comparing a currently captured image of the segment of skin (block 914) with an image of the same segment of skin, retrieved from processor 104 memory, captured prior to the current procedure or during a previous procedure and stored in memory of the mobile phone or tablet computer processor 104 (block 924). Information extracted from the comparison could be integrated and processed with the parameters described above (block 928).

It will also be appreciated by persons skilled in the art that the present method and system are not limited to what has been particularly shown and described hereinabove. Rather, the scope of the method and system includes both combinations and sub-combinations of various features described hereinabove as well as modifications and variations thereof which would occur to a person skilled in the art upon reading the foregoing description and which are not in the prior art.

1-21. (canceled)

22. System for personal skin procedure comprising:
a mobile phone or tablet computer (100) with a camera (102);
an electromagnetic energy emitting home-use cosmetic skin treatment device (114) operative to emit electromagnetic energy and including at least controller (116);
a communication link between at least the mobile phone (100) and the home-use cosmetic skin treatment device (114); and
a patch (500, 600, 700) adhereable to skin of a user, the patch including at least one of a time code (302) scale bar (304), a scale frame (306), a numerical image identifier (308, 310) and skin pigmentation references (402).

23. The system according to claim 22, wherein the mobile phone or tablet computer also includes a processor running a cosmetic skin procedures software application (App) configured to
capture an image of a segment of skin;
analyze and process the image and extract from the image information regarding the segment of skin;
determine specific optimal procedure doses of electromagnetic or mechanical energy for one or more skin fractions within the segment of skin based on the extracted information; and
change settings of electromagnetic or mechanical energy emitting home-use cosmetic skin treatment device based on a determined specific optimal procedure doses of electromagnetic energy.

24. The system according to claim 22, wherein the patch is opaque, semi-opaque or transparent.

25. System for personal skin procedure comprising:
at least one portable computer with a camera;
at least one software application (App) run on the computer;
at least one electromagnetic energy emitting home-use cosmetic treatment device;
at least one communication link between the portable computer and the cosmetic treatment device; and
a patch to a segment of skin desired to be treated, the patch including at least one of a time code, a scale bar, a scale frame, a numerical image identifier and skin pigmentation references; and
wherein the App employs the computer to
analyze and process an image of a segment of skin and extract from the image of the segment of skin and the patch information regarding the segment of skin;
determine specific optimal procedure doses of light (optical) energy for one or more skin fractions within the segment of skin based on the extracted information; and at least one of
change settings of a light energy emitting home-use cosmetic treatment device based on the determined specific optimal procedure doses of light (optical) energy; and
activate a light energy emitting home-use cosmetic treatment device based on the determined specific optimal procedure doses of light (optical) energy.

26. The system according to claim 25, wherein the App also identifies an inputted cosmetic device and selects a corresponding display.

27. The system according to claim 26, wherein the App also communicates a raw image captured by the camera to a remote computer; and
based on a determined specific optimal procedure doses of light (optical) energy changes settings of an identified home-use cosmetic treatment device; and/or
activates the identified home-use cosmetic skin treatment device.

28. Method for capturing an image of a segment of skin covered by hair a mobile phone or a tablet computer camera comprising:
applying a patch to a segment of skin desired to be treated, the patch including at least one of a time code, a scale bar, a scale frame, a numerical image identifier and skin pigmentation references;
capturing an image of a segment of skin desired to be treated and of the patch;
analyzing and processing the image and extracting from the image information regarding one or more parts of the segment of skin; and
determining specific optimal procedure doses of electromagnetic or mechanical energy for one or more skin fractions within the segment of skin based on the extracted information.

29. The method according to claim 28, wherein information regarding the one or more parts of the segment of skin includes at least one parameter selected from a group of parameters consisting of density of hair on the segment of skin, diameter of individual hairs, pigmentation of hair, pigmentation of skin and location of individual hairs on the segment of skin.

30. The method according to claim 29, wherein parameters also include pigment parameters of a desired area to be treated and parameters of electromagnetic energy to be applied to the segment of skin.

31. The method according to claim 30, also comprising
using a mobile phone or tablet computer camera for
capturing an image of a segment of skin; and
changing settings of an electromagnetic energy emitting home-use cosmetic skin treatment device based on a determined specific optimal procedure doses of electromagnetic energy; and
activating a home-use cosmetic skin treatment device.

32. The method according to claim 29, also comprising using the mobile phone or tablet computer processor to communicate the specific optimal procedure doses for cosmetic procedure of the segment of skin and corresponding to a captured image of a segment of skin desired to be treated and of the patch to a home-use cosmetic skin treatment device.

33. The method according to claim 28, further comprising using a mobile phone or tablet computer processor to determine the specific optimal procedure doses for a cosmetic procedure on the segment of skin in an image of a segment of skin desired to be treated and of the patch captured by a camera.

34. The method according to claim 33, also comprising using the mobile phone or tablet computer processor to communicate the specific optimal procedure doses for cosmetic procedure of the segment of skin and corresponding to a captured image of a segment of skin desired to be treated and of the patch to a home-use cosmetic skin treatment device.

35. The method according to claim 28, also comprising displaying an image of the one or more parts of the segment of skin and of the patch on a display and storing the image in memory of a mobile phone or tablet computer.

36. The method according to claim 28, also comprising monitoring progress of a cosmetic procedure by comparing a currently captured image of the segment of skin with an image of a same segment of skin captured prior to application of the current procedure or during a previous procedure and stored in a memory of a mobile phone or tablet computer.

37. The system according to claim 22, wherein the numerical identifier is at least one of a serial number (308) and a barcode (310).
